(19) United States
(12) Patent Application Publication
(10) Pub. No.: US 2014/0261439 A1
(43) Pub. Date: Sep. 18, 2014

(54) Sterile Oxygen And Anesthesia Mask Holder

(71) Applicant: SYNAPTIC WIRELESS, LLC, O'Fallon, MO (US)

(72) Inventors: Charles H. Bowen, O'Fallon, MO (US); P. Mark Bowen, Fayetteville, GA (US)

(73) Assignee: SYNAPTIC WIRELESS, LLC, O'Fallon, MO (US)

(21) Appl. No.: 13/797,306

(22) Filed: Mar. 12, 2013

(51) Int. Cl. A61M 16/06

(52) U.S. Cl. CPC A61M 16/0683
USPC 128/206.22

(57) ABSTRACT

A holder for temporarily retaining a mask and directing condensed moisture draining from such a mask away from the mask and into an isolating structure such as a reservoir. Among other things, this allows a mask to be quickly placed in a stable location without contaminating the mask and without the mask contaminating another surface.

### BACKGROUND

1. Field of the Invention

This disclosure is related to the field of medical gas masks, specifically, oxygen and anesthesia masks which must be temporarily stored in sanitary fashion during medical procedures.

2. Description of the Related Art

Masks are commonly used in medical procedures. For example, an oxygen mask is used to provide breathing oxygen from a tank or canister to a patient's lungs. Similarly, anesthesia masks direct anesthetic gases to and from the patient. Outside of the medical field, oxygen masks are used in other contexts, such as by military pilots and commercial airline passengers during a loss of cabin pressure, as well as SCUBA divers and high-altitude climbers.

These masks share several features. First, they generally fit over the mouth and/or nose and are secured to the wearer by a retention system. In medicine, this retention system is generally a set of elastic bands attached to the mask and placed behind the patient's head to secure the mask to the patient's face. In other contexts, other fastening systems are used, such as by attaching to the flight helmet for pilots. Second, the masks are connected to an oxygen supply, usually by a flexible ribbed hose connected to the mask at one end and to the oxygen supply at another. The ribbed hose can bend without developing a kink that could impede the flow of gas. Some masks have multiple hoses, such as an anesthesia mask, for example, to carry away respiratory byproducts or prevent the dangerous buildup of gas. In some contexts, the connection of the mask to the face must be secure but need not be airtight. Indeed, some masks have holes which provide ventilation to the ambient atmosphere.

As with most medical devices, it is important that the mask be sanitized and/or sterilized, particularly where a mask is used in an operating room, as the presence of microbes poses a risk of infection and/or disease to the patient, physician and staff. While masks are usually sterilized prior to use, maintaining sanitary masks during the procedure is difficult. The mask may need to be temporarily removed, such as to intubate the patient, and the person holding the removed mask may need both hands free to perform other tasks. The mask generally should not be set down on a nearby surface. For one, the mask itself tends to be lightweight, and the drag of the hose tends to pull the mask off the surface, depositing it onto the floor or another surface, and potentially contaminating the mask. It must then be resanitized/resterilized or replaced to avoid risking contamination of the patient. However, in a surgical environment, time is short and access to continuous oxygen flow is crucial. Even a short delay while a new mask is attached poses a risk of oxygen deprivation.

The mask itself also becomes contaminated by contact with the patient and his respiratory byproducts. As the patient breaths through the mask, warm air from the patient transfers heat and moisture to the mask, warming its surface and causing moisture to accumulate in the mask. Operating rooms tend to be kept at relatively low temperatures to discourage the development of microbes. When the mask is removed from the patient and set on a cold surface, the mask cools and condensation may dew off the mask and drip unto the surface. Contaminants, such as microbes caused by the patient's respiration into the mask may be transferred to the surface through the condensation, contaminating the surface. These contaminants may then further contaminate other tools later placed on the surface.

### SUMMARY

The following is a summary of the invention in order to provide a basic understanding of some aspects of the invention. This summary is not intended to identify key or critical elements of the invention or to delineate the scope of the invention. The sole purpose of this section is to present some concepts of the invention in a simplified form as a prelude to the more detailed description that is presented later.

Because of these and other problems in the art, there is described herein a holder for temporarily retaining a mask and directing condensed moisture draining from such a mask away from the mask and into an isolating structure such as a reservoir. Among other things, this allows a mask to be quickly placed in a stable location without contaminating the mask and without the mask contaminating another surface.

There is described herein, among other things, an oxygen mask holder comprising: a base having a top surface oriented at an angle with respect to the pull of gravity such that the top surface has a low end and an opposing high end; a generally planar mask film having a high friction surface and an opposing back, the mask film sized and shaped for placing an oxygen mask on the mask film; wherein the back of the mask film is on the top surface and an oxygen mask can be placed on the high friction surface; wherein the angle is such that when there is liquid on the top surface the liquid will flow toward the low end.

In an embodiment, the holder further comprises a reservoir positioned such that when liquid on the mask film flows toward the low end, the liquid flows into the reservoir.

In an embodiment of the holder, the mask film is part of a pad of similar mask films.

In an embodiment of the holder, the mask film includes an anti-microbial additive.

In an embodiment of the holder, the anti-microbial additive comprises at least one item selected from the group consisting of: an anti-fungal, an anti-viral, and an anti-bacterial.

In an embodiment of the holder, the base is generally in the configuration of a triangular prism.

In an embodiment of the holder, the angle is between about 30 degrees and about 60 degrees.

In an embodiment of the holder, the angle is about 60 degrees.

In an embodiment of the holder, the holder further comprises a clip sized and shaped for accepting an oxygen mask hose and attached to the base near the low end.

In an embodiment of the holder, the high friction surface comprises an adhesive.

There is also described herein, a method for sanitarily storing an oxygen mask during a medical procedure comprising the steps of: providing an oxygen mask holder comprising: a base having a top surface oriented at an angle with respect to the pull of gravity such that the top surface has a low end and an opposing high end; a mask film having a high friction surface and an opposing back, the mask film sized and shaped for placing an oxygen mask on the mask film; wherein the back of the mask film is on the top surface and an oxygen mask can be placed on the high friction surface; and wherein the angle is such that when there is liquid on the top surface the liquid will flow toward the low end; providing an oxygen mask comprising a face mask and a hose attached thereto; the patient breathing into the face mask as part of a medical procedure; removing the face mask from the patient; placing the face mask on the mask film; holding the face mask in place with the mask film.

In an embodiment the method further comprises: providing a reservoir positioned such that when liquid on the mask film flows toward the low end, the liquid flows into the reservoir; liquid drained onto the mask film from the oxygen mask flowing toward the low end; liquid drained onto the mask film flowing into the reservoir.

In an embodiment of the method, the mask film is part of a pad of similar mask films.

In an embodiment of the method, the mask film includes an anti-microbial additive.

In an embodiment of the method, the anti-microbial additive is selected at least one item selected from the group consisting of: an anti-fungal, an anti-viral, and an anti-bacterial.

In an embodiment of the method, the angle is between about 30 degrees and about 60 degrees.

In an embodiment of the method, the angle is about 60 degrees.

In an embodiment of the method, the base is generally in the configuration of a triangular prism.

An embodiment of the method further comprises: providing a holder further comprising a clip sized and shaped for accepting an oxygen mask hose and attached to the base near the low end; clipping the hose with the clip when the mask is placed on the mask film.

In an embodiment of the method, the high friction surface comprises an adhesive.

### BRIEF DESCRIPTION OF THE DRAWINGS

FIG. 1 provides a perspective view of a first embodiment of a mask holder with a mask thereon.

FIG. 2 provides a perspective view of a mask holder including a hose retaining clip.

FIG. 3 provides a side view of a second embodiment of a mask holder with a mask thereon.

FIG. 4 provides a side view of a third embodiment of a mask holder having two pads.

### DESCRIPTION OF THE PREFERRED EMBODIMENTS

This disclosure teaches by way of example and not limitation. While the present disclosure is described primarily in terms of an oxygen or anesthesia mask used in an operating room, one of ordinary skill in the art will understand this disclosure to include other applications, including but not limited to other medical devices which may gather moisture or need to be temporarily placed on a sterile surface, and other structures with members to accommodate such devices.

FIG. 1 depicts an embodiment of a mask holder (101) with a mask (109) disposed thereon. The depicted embodiment includes a base (103) having a top surface (105), a high friction mask film (107) placed on the top surface (105), and a reservoir (115).

The depicted base (103) of FIG. 1 is in the configuration of a triangular prism with a rectangular top surface (105). Although a generally right triangular prism is depicted, in an alternative embodiment, the base (103) is in the configuration of an isosceles or scalene triangular prism. An embodiment with a scalene triangular base is depicted in FIG. 4 without a mask (109) thereon. Among other things, the embodiment of FIG. 4 provides two top surfaces (105a) and (105b) for two mask films (107a) and (107b). This allows for a quick switch between films in the event that a first surface is soiled or otherwise unusable. Further, in an embodiment having a scalene triangular prism base (103), the scalene shape provides two different top surfaces, a smaller surface (105a) and a larger surface (105b), each having two different angles (117a) and (117b), on which a mask film (107a) and (107b) may be placed, allowing the holder (101) to accommodate multiple mask (109) designs. Such an embodiment has the advantage of presenting two opposing top surfaces (105a) and (105b) which can each support a mask providing increased functionality between different masks, or allowing for multiple masks to be simultaneously placed for the same patient. In a still further embodiment, the base (103) may be a non-triangular polygonal prism.

The top surface (105) of the base (103) is generally planer and oriented at an angle (117) relative to the pull of gravity (113). It is generally preferred that this angle (117) be greater than 45 degrees with respect to gravity (113) or, said another way, less than 45 degrees with respect to the horizontal—i.e., a planar surface perpendicular to gravity (113). In an embodiment, the angle (117) is between about 30 degrees and about 60 degrees and may be about 45 or about 60 degrees. The angle (117) is preferably steep enough that runoff from the mask (109) will flow down the top surface (105) or a mask film (107) thereon and drain into the reservoir (115), but shallow enough that the friction between the mask (109) and the mask film (107) is stronger than the pull of gravity (113) on the mask (109). The precise angle (117) may vary and, in an embodiment, the angle may be adjustable through any means known in the art or in the future developed.

The base (103) may be constructed of any material sufficiently rigid to maintain its own shape and support the weight of the other components, and will generally be made from a sanitizable or sterilizable material suitable for use in a medical environment, including but not limited to a medical plastic and stainless steel.

In an alternative embodiment, such as depicted in FIG. 3, the base (103) includes a generally a rectangular prism (102) attached to an elongated member (104). In such an embodiment, the elongated member (104) of the base (103) is configured for attaching to a surface in the environment. In such an embodiment, the elongated member (104) will generally be attached to a stable surface through an attaching means, which will necessarily vary from embodiment to embodiment, depending upon where the support is to be attached. By way of example and not limitation, in an embodiment the attaching means includes a weighted base attached to the bottom of the elongated member (104), a clamp, a friction or tension mount, a slot or pin for connecting to a related structure, a magnet, an adhesive, or other support structures. In a still further embodiment, the attaching means includes an elongated member (104) being sized and shaped or otherwise configured for fitting into a coupling sized and shaped for accepting the elongated member (104), including but not limited to a generally cylindrical pocket or sleeve. In a further embodiment, the coupling is attached to other equipment in the operating room. In a still further embodiment, the support is secured via hardware, including but not limited to: a bolt, dowel, or a circular clamp.

Although the depicted support of FIG. 3 is a vertical element attached to the bottom side of the base (103), this is just one possible way the components may be arranged and/or interconnected. In an alternative embodiment, the support is a lateral element. In a still further embodiment, the support attaches to the top or side of the base (103). The support also need not be a single, cylindrical element, but may be of another shape or size, including an elongated polygonal prism. In an embodiment, there is a plurality of members (104) which may be “legs.” In a still further embodiment, the support is removable from the base (103). It is specifically contemplated that the support may comprise any means of providing or supporting the angled (119) orientation of the top surface (105). The support may be affixed to the base (103) through any means known in the art for connecting these types of components, including but not limited to monolithic construction, hardware, adhesive, interlocking threads, and static fit.

In the depicted embodiments of FIGS. 1-2, the top surface (105) is a generally rectangular surface sized and shaped to allow an oxygen mask (109) to be placed upon it or upon a mask film (107) on the top surface (105). The top surface (105) is generally rigid and generally planar.

In the embodiment of FIG. 1, the angle (117) is achieved by sizing and shaping the base (103) such that the top surface (105) is oriented at the desired angle (117) relative to gravity when the base (103) is at rest on a generally flat planar surface which is itself generally perpendicular to gravity, such as a table, counter, or other equipment found in a medical facility. In the embodiment of FIG. 3, the angle (117) is achieved by attaching the rectangular prism (102) to elongated member (104) at the desired angle (117), and securing member (104) such that member (104) is generally parallel with the pull of gravity (113). The angular (117) orientation allows condensation and other material to runoff from the mask (109) and be directed away from the mask (109), rather than pool or collect near it.

Although a generally rectangular top surface (105) is depicted, the top surface (105) is not confined to this shape. Generally, the shape and size of the top surface (105) will be larger than the longest lateral and anteroposterior dimensions of the oxygen mask (109) with which the holder (101) will be used. This is because, among other things, the top surface (105) primarily provides support for the high friction mask film (107), and the preferred dimensions of the mask film (107) are larger than those of the oxygen mask (109), so that the mask film (107) presents a large enough surface for attaching a mask (109) so that a doctor or other staff can quickly place the mask (109) on the mask film (107) without much need for precision.

In the depicted embodiment, the mask film (107) is a high friction surface, similar to mats used in conjunction with clean rooms and other areas where it is desirable to remove material attached to footwear before entering the room. These pads generally have a tacky surface which traps or removes dirt, dust, and debris from footwear as persons walk over the mats. These mats are often placed outside of entranceways to sensitive areas. Generally the high friction surface includes a tacky adhesive which does not transfer from the pad to shoes. In a further embodiment, the mask film (107) includes an acrylic adhesive. In the depicted embodiment, the mask film (107) is placed on the top surface (105), with the high friction side facing upward. The mask film (107) may also be attached or removably attached to the top surface (105). In an alternative embodiment, the mask film (107) is part of a pad or stack of similar films (107), which may have a backing potentially connecting the films (107) together. In such an embodiment, the film backing or back layer of film (107) in the pad is attached or placed upon the top surface (105). In such an embodiment, the mask film (107) on top the pad is used for an operation and, when the operation concludes, the spent film (107) may be removed from the stack and discarded, presenting the next film (107) in the stack for use in the next procedure, which is still sanitized or sterilized. When the entire pad of mask films (107) is exhausted, a new pad may be attached to the top surface (105). In an embodiment, the mask film (107) is comprised of polyethylene film. In an embodiment, this surface is between one-sixteenth ( 1/16) and one quarter (¼) of an inch thick. In a still further embodiment, the mask film (107) includes an anti-microbial additive, including but not limited to: an anti-bacterial, an anti-viral, and/or, an anti-fungal.

The mask film (107) generally is oriented parallel to the top surface (105), causing the mask film (107) to have generally the same angular (117) orientation with respect to gravity (113) as does the top surface (107). This facilitates the draining of runoff from the mask (109) into the reservoir (115).

When an oxygen mask (109) is removed from a patient to be stored temporarily, the mask (109) is placed on the mask film (107) with the open end of the mask (109) facing the mask film (107). The high friction surface holds the mask (109) in place and the tacky adhesive surface attracts dust and other debris off the mask (109).

The mask (109) will generally warm while attached to the patient, as both the patient's skin and respiratory byproducts transfer heat to the mask (109). The patient's exhalation also generates moisture and humidity within the mask (109). When the mask (109) is removed and placed on the mask film (107), the mask (109) will begin to cool, causing humidity and moisture remaining in the mask's (109) cavity to begin to condense and drain onto the mask film (107). The angled (117) orientation will then cause runoff to drain off the mask film (109), preventing it from pooling near the mask (109) and creating a further contamination risk. The precise angle (117) may vary and, in an embodiment, the angle may be adjustable through any means known in the art or in the future developed. As the film (107) is also preferably anti-microbial, the action of the runoff flowing over the film (107) exposes a relatively greater percentage of the runoff to the film (107), increasing the effectiveness of the anti-microbial agent.

In an embodiment, the mask film (107) or top surface (105) includes channels which direct runoff to flow through a defined pathway. For example, a V-shaped channel may direct runoff towards a single point of egress from the mask film (107) or top surface (105). In an embodiment, the channels direct the runoff through a filter or filtration system, which in turn may include anti-microbial agents, to further remove contaminants from the runoff.

In an embodiment, the holder (101) further includes a system for isolating and storing runoff from the mask (109). In the depicted embodiment of FIG. 1, this system is a reservoir (115) located beneath the lower end of the mask film (107) so that liquid running down the angled (117) film (107) or the top surface (105) will flow toward and drain into the reservoir (115). The reservoir (115) may be a pool or basin built into the base (103) or removably attached to the base (103), or a separate member placed near the base (103), or which the base (103) is placed near. In an embodiment, the reservoir (115) includes a hose, tube, or other means (not depicted) for transferring captured runoff to a drain or other disposal system. In a still further embodiment, the reservoir (115) includes a cavity within a component of the holder (101), such as but not limited to the base (103). In an depicted embodiment, the reservoir (115) is in the shape of a trough, but any size and shape suitable for use with the base (103) is contemplated. For example, in the depicted embodiment of FIG. 3, the reservoir (115) is in the shape of a bowl. In a further embodiment, the system for isolating and storing runoff includes an absorbing or adsorbing means, such as a sponge or desiccant (not depicted) to aid in cleaning and disposal.

In an embodiment, the holder (101) includes systems for further securing the mask (109) and/or for managing the hose (111). The mask (109) is a relatively lightweight component attached to a hose (111), and the length of hose (111) is generally long enough that if the mask (109) is not held with sufficient force, the drag of the hose (111) may pull the mask (109) off the holder (101). The high friction surface of the mask film (107) provides sufficient force to secure the mask (109) in many applications, but it may be desirable in some applications to include other systems for further securing the mask (109).

In an embodiment, the holder (101) further includes a mask clip (201) as depicted in FIG. 2. The depicted clip (201) is a flexible element in the shape of a partial circle having a diameter slightly greater than that of the hose (111) and with an open top that can flex so that the opening is large enough for the hose (111) to pass through and “snap” into position such that the clip (201) partially circumscribes the hose (111). When the mask (109) is placed on the mask film (107), the hose (111) is positioned over the clip (201) and can be quickly snapped into it. This not only provides an additional point of security for the mask (109) which, if it is bumped or otherwise disturbed, will not simply tumble to the floor because the clip (201) holds the hose (111) which is attached to the mask (109) in place, but also provides for management of the hose (111) which otherwise may swing while suspended from the mask (109) or hang at an inconvenient or obstructive orientation. The depicted clip (201) is attached to the surface (105) of the base (103) but a clip (201) may be attached to any portion of the holder (101) or to another piece of equipment in the operating room. In an embodiment, the holder (101) includes multiple clips (201) attached to the components of the holder (101) as necessary to direct the hose (111) away from the surgical field.

In FIG. 3, the fastener (119) which holds the mask (109) to the patient's face will generally hang free when the mask (109) is on the mask film (107). In some applications, this could cause the fastener (119) to interfere with the mask's (109) connection to the mask film (107), weakening the connection and increasing the likelihood that the mask (109) may become dislodged from the holder (101). For example, where the fastener (119) is an elastic band connected to the mask (109) at opposing lateral edges, when the mask (109) is placed on the mask film (107), the fastener (119) should be moved out of the way so it is not trapped between the mask (109) and mask film (107). Placing the mask (109) in this fashion may take longer than desired. In an embodiment, the holder (101) includes a means for managing the fastener (119), which allows the mask (109) to be quickly placed on the mask film (107) without the interference of the fastener (119). By way of example and not limitation, the holder (101) may include a small hook or pin (not depicted) attached to the higher elevated end of the top surface (105). When the mask (109) is placed on the mask film (107), the fastener (119) is draped over this hook or pin to move it out of the way.

In another embodiment, this means may be an existing component. For example, in the depicted embodiments of FIGS. 1 and 3, when the mask (109) is placed, the doctor holds the mask (109) just beyond the elevated portion of the top surface (105) and lowers the mask (109) until the loop of the fastener (119) is behind the back edge the backing base (103). The doctor then pulls the mask (109) toward the lower end of the top surface (105) so that the mask (109) is above the mask film (107), causing the fastener to loop around the back of the base (103). Then, when the mask (109) is placed on the mask film (107), the fastener (119) is out of the way.

In a further embodiment, the components are sized and shaped so that the mask (109) is inhibited from sliding or falling off the mask film (107) if the fastener (119) is disposed as described. In such an embodiment, the tension on the elastic fastener (119) when looped behind the base (103) is such that if the mask (109) begins to fall or slide down the inclined pad (107), the fastener (119) tightens to the point that the mask (109) will generally discontinue sliding before reaching the low side of the mask film (107) or top surface (105), and thus the fastener (119) inhibits the mask (109) from falling.

In an embodiment, the holder (101) is used by placing a mask (109) which has or will be used in conjunction with medical treatment of a patient on a sterile and/or sanitized mask film (107) and retrieving the mask therefrom when the mask (109) is to be placed on the patient again. The mask film (107) may be changed between placements of the mask (109), or may be re-used during the course of a procedure and removed for the next procedure. In an embodiment including a pad, the removed mask film (107) reveals a fresh, sanitized and/or sterilized film (107) in the pad. In an embodiment having a reservoir (115), the reservoir (115) may also be replaced, sanitized or sterilized during or between procedures.

While the systems, devices, and methods have been disclosed in connection with certain preferred embodiments, this should not be taken as a limitation to all of the provided details. Modifications and variations of the described embodiments may be made without departing from the spirit and scope of this invention, and other embodiments should be understood to be encompassed in the present disclosure as would be understood by one of ordinary skill in the art.

1. An oxygen mask holder comprising:
a base having a top surface oriented at an angle with respect to the pull of gravity such that said top surface has a low end and an opposing high end;
a generally planar mask film having a high friction surface and an opposing back, said mask film sized and shaped for placing an oxygen mask on said mask film;
wherein said back of said mask film is on said top surface and an oxygen mask can be placed on said high friction surface;
wherein said angle is such that when liquid is on said top surface said liquid will flow toward said low end.

2. The holder of claim 1, where said holder further comprises a reservoir positioned such that when liquid on said mask film flows toward said low end, said liquid flows into said reservoir.

3. The holder of claim 1, wherein said mask film is part of a pad of similar mask films.

4. The holder of claim 1, wherein said mask film includes an anti-microbial additive.

5. The holder of claim 4, wherein said anti-microbial additive comprises at least one item selected from the group consisting of: an anti-fungal, an anti-viral, and an anti-bacterial.

6. The holder of claim 1, wherein said base is generally in the configuration of a triangular prism.

7. The holder of claim 1, wherein said angle is between about 30 degrees and about 60 degrees.

8. The holder of claim 1, wherein said angle is about 60 degrees.

9. The holder of claim 1, wherein said holder further comprises a clip sized and shaped for accepting an oxygen mask hose and attached to said base near said low end.

10. The holder of claim 1, wherein said high friction surface comprises an adhesive.

11. A method for sanitarily storing an oxygen mask during a medical procedure comprising the steps of:
providing an oxygen mask holder comprising:
a base having a top surface oriented at an angle with respect to the pull of gravity such that said top surface has a low end and an opposing high end;
a mask film having a high friction surface and an opposing back, said mask film sized and shaped for placing an oxygen mask on said mask film;
wherein said back of said mask film is on said top surface and an oxygen mask can be placed on said high friction surface; and
wherein said angle is such that when liquid is on said top surface said liquid will flow toward said low end;
providing an oxygen mask comprising a face mask and a hose attached thereto;
said patient breathing into said face mask as part of a medical procedure;
removing said face mask from said patient;
placing said face mask on said mask film;
holding said face mask in place with said mask film.

12. The method of claim 11, wherein said method further comprises:
providing a reservoir positioned such that when liquid on said mask film flows toward said low end, said liquid flows into said reservoir;
liquid drained onto said mask film from said oxygen mask flowing toward said low end;
liquid drained onto said mask film flowing into said reservoir.

13. The method of claim 11, wherein said mask film is part of a pad of similar mask films.

14. The method of claim 11 wherein said mask film includes an anti-microbial additive.

15. The method of claim 14, wherein said anti-microbial additive is selected at least one item selected from the group consisting of: an anti-fungal, an anti-viral, and an anti-bacterial.

16. The method of claim 11, wherein said angle is between about 30 degrees and about 60 degrees.

17. The method of claim 16, wherein said angle is about 60 degrees.

18. The method of claim 11, wherein said base is generally in the configuration of a triangular prism.

19. The method of claim 11, wherein said method further comprises:
said providing a holder further comprising a clip sized and shaped for accepting an oxygen mask hose and attached to said base near said low end;
clipping said hose with said clip when said mask is placed on said mask film.

20. The method of claim 11, wherein said high friction surface comprises an adhesive.
